(19) United States
(12) Patent Application Publication
(10) Pub. No.: US 2023/0194774 A1
(43) Pub. Date: Jun. 22, 2023

(54) OPTICAL WAVEGUIDE AND METHOD OF FABRICATION THEREOF

(71) Applicant: HAUTE ECOLE ARC, Neuchâtel (CH)

(72) Inventors: Miguel LLERA, La Chaux-de-Fonds (CH); Frédéric FLAHAUT, Morteau (FR); Sylvain BERGERAT, Le Russey (FR)

(21) Appl. No.: 17/924,981

(22) PCT Filed: May 13, 2020
(86) PCT No.: PCT/EP2020/063320

§ 371 Date: Nov. 11, 2022

(51) Int. Cl. G02B 6/02; G02B 1/04

(52) U.S. Cl. CPC G02B 6/02033; G02B 1/048

(57) ABSTRACT

Disclosed is an optical waveguide, for transmitting a guided optical light beam having a wavelength greater than 180 nm. The waveguide includes a core layer for guiding light made of a first material having a first index of refraction, and a cladding layer made of a thermoplastic elastomer. Also disclosed are: a medical device and also to a waveguide sensor including the optical waveguide of the invention; a method of fabrication of the optical waveguide. The method includes a step of providing a thermoplastic elastomer preform having a central longitudinal aperture for introducing a liquid polymer, before or after reducing and elongating the preform to a predetermined length and lateral dimension. The method includes a polymerizing step of the core of the formed optical waveguide; and use of the optical waveguide in association with a surgical instrument.

### TECHNICAL FIELD OF THE INVENTION

The present invention relates to the field of optical waveguides. The present invention more specifically relates to flexible optical waveguides such as optical fibers or flat waveguides that may be used advantageously in applications and devices wherein it is difficult to provide light to a distant target and in which the light path may be tiny, present short curvatures and/or complex shape. The invention relates to flexible optical waveguides, that are biocompatible and stretchable and are based on the use of elastomers. The invention proposes a solution to applications wherein breakage of an optical waveguide would have dramatic consequences.

### BACKGROUND OF THE INVENTION

Optical waveguides constitute means to provide light to a distant target and may be deployed over long lengths and through narrow spaces and possibly harsh environments. Optical waveguides in the form of fiber optics, fiber bundles or flat waveguides have been developed for a wide range of applications such as telecom, industrial and medical applications. In the case of telecom applications, the focus has been put into processes that allow to provide extremely low absorption over very long lengths, and mainly in the infrared. Other applications such as industrial machines or medical application usually do not have this requirement but have other requirements such as their mechanical properties or also compatibility requirements which are mandatory in chemical, bio-chemical or medical environments. In the case of medical implants for example, the bio -compatibility is a main requirement. This bio - compatibility is mostly also linked to other requirements such as mechanical security requirements

For example, glass optical fibers for human implants face many difficulties as biocompatibility or breakability that can be dramatic in such applications. Furthermore, implants fabrication often needs highly flexible optical waveguides because of the complex fabrication steps of the implants.

Most existing optical waveguides are based on glass or plastic fibers and are not suitable for some medical applications such as implants.

Several attempts have been made in the past to realize optical fibers having improved mechanical properties and/or their medical or biochemical compatibility. For example, polyurethane (PU) in optical fibers field has been widely used in the coating design and the fiber optic tubing for cable production.

Several solutions have been proposed to improve the mechanical properties of bent fibres. For example, documents JPS59111952 and WO2003091178A2 propose to use polyurethanes to increase the adhesivity of the coating on the glass fiber and to protect the fibre from the micro-bending effects. PU has also been used to reinforce optical fibers in order to increase their corrosion and weather resistance in aerial optical cables, as described in CN107589507. Also, the use of PU for better resistance of optical fibers for medical applications has been described in for example CN206431340. Also, the use of PU in complex coating structures in order to correct the defects on primary coatings has been proposed in US2013243948.

In the field of fiber-optic protection-tubing applications, which deals with the fabrication of optical cables, polymers such as PU has been proposed in CN203275734, solely as an additional component to increase the cable flexibility.

Other documents in the field of optical fibres propose the use of PU as unclad optical fibres. For example, US4915473A discloses a pressure sensor by using a PU fiber whose optical transmission is inversely proportional to the pressure applied on it. Also, US20080089088A1 describes to use an unclad PU fiber to produce a side scattering for lightning and decorative applications

Flexible optical fibers for in-vivo use in the tissue of a living mammal is described in US4893897. The document US4893897 describes a fabrication process in which two materials, such as polystyrene and aliphatic PU, are used to produce the core and the cladding of the fiber. The process is based on the melting and co-extruding of the two materials to produce a fiber-like preform which is finally drawn to the required final optical fiber dimensions. A main constraint of the fabrication of US4893897 is that the cladding material must have a melt viscosity lower than the one for the core.

Another document JP 62269905 describes how to produce a flexible optical fiber by injecting a liquid PU resin onto a hollow flexible fiber and then by polymerizing this liquid resin with a UV light. The photocurable liquid resin is for example polyurethane poly (meth) acrylate alone but could also be a monovinyl compound such as alkyl (meth) acrylate or other materials. For the hollow fiber production, the materials used were polytetrafluoroethylene, ethylene-vinyl acetate copolymer, vinyl chloride resin, and other kind of materials. The hollow fiber must be extruded by using a concentric annular shaped die. Then the liquid resin is pushed on one side and sucked by using a vacuum pump on the other side of the hollow fiber. The polymerization of the liquid core is performed by UV light. The hollow fiber production generates very low-quality surfaces that leads to huge absorption effects and so to unacceptable optical transmissions.

Early use of optical silicone to produce optical fibers was described in the document JPS5447667 that describes a glass core covered with a silicone cladding layer. The use of silicone as a coating of glass optical fibers is described in several documents such as JPS6230152 and JPH01286939, CN108977069. Even with a silicone cladding these fibers remain unacceptable for several applications such as medical implants.

Polymer fibers such as silicone fibers have been described in for example US5237638A. The fabrication of such polymer fibers is realized by dipping an extruded core into a cladding solution and then by curing the cladding. Liquid silicone can be used to produce fluid light guides, as described in US5692088A. Such liquid silicone waveguides use a flexible tube with a specific film fixed at the internal surface to play the cladding role while the core is a liquid polymer as a fluid silicone. This technique can be used to produce liquid core flexible catheters for laser ablation, as described in US9700655B2. Silicone light guides use non-curable liquid silicone and the cladding is realized by a specific treatment on the internal tube surface and the guide sizes are on another order of magnitude. Such optical fibers are limited to cores that have a large lateral cross section and the production process is difficult to reproduce relative to the required optical properties. Also, these kinds of optical fibers are only used for light delivery systems where the core diameter is less important. For sensing applications, a single-mode operation is more suitable.

Highly stretchable optical fibers are described in US2012244143 and CN107907484. The document US2012244143 proposes the use of silk while the documents CN107907484 and US2016177002 describe the use of hydrogel. Approaches based on silk or hydrogels are not useful for different applications or configurations such as the use of them to interrogate optically resonating cavities such as Fabry-Perot cavities arranged at the end of a waveguide.

There is thus a need for improved waveguides, such as optical fibers, because existing waveguides that are based on glass or plastic fibers are not suitable for a wide range of applications, such as implants or other medical applications.

### SUMMARY OF THE INVENTION

The inventors of the present invention have found solutions to the above-discussed problems by providing optical waveguides, such as optical fibers, having a cladding made of an elastomer, preferably a thermoplastic elastomer (TPE) such as a polyurethane. The fabrication process of the fibers and waveguides of the invention provides a wide range of advantages such as the decrease of the inherent complexity of optical waveguide production to a level where implant manufacturers could, at least partially, produce their own optical waveguides.

More precisely the invention is achieved by an optical waveguide , comprising a core layer, defining a longitudinal axis Z, and a cladding layer surrounding said core layer. The core layer and the cladding layer are configured to transmit along said longitudinal axis Z a light beam having a wavelength greater than 180 nm. The core layer is made of a first material having a first index of refraction n1 and the cladding layer is made of at least one layer made of a thermoplastic elastomer (TPE) having a second index of refraction n2 being smaller than said first index of refraction n1.

In an embodiment said at least one layer of thermoplastic (TPE) is one of: a styrenic block copolymer (TPE-s), thermoplastic polyolefinelastomers (TPE-o), thermoplastic Vulcanizate (TPE-v or TPV), thermoplastic polyurethanes (TPU), thermoplastic copolyester (TPE-E), thermoplastic polyamides (TPE-A) or not classified thermoplastic elastomers,(TPZ), In an embodiment the core layer is made of a polymer, possibly silicone.

Preferably said thermoplastics is a thermoplastic as defined according to the ISO norm 18064.

In embodiments the waveguide is optical fiber, possibly a monomode optical fiber. In variants, the optical waveguide has a first lateral side having a first width W1 and a second side having a second width W2 larger than said first width W1, said widths (W1, W2) being defined in any lateral cross section, defined in an plane (X-Y) orthogonal to said longitudinal axis Z.

In an particular realization the core layer may have an index of refraction of 1, and the inner surface of said cladding layer may comprises a metallic and/or dielectric layer arranged to reflect light that is incoupled into said core layer..

In embodiments the optical waveguide is configured to guide less than 100 modes, preferably less than 20 modes, more preferably less than 5 modes, defined in at least one longitudinal plane (X-Z, Y-Z). In variants, optical waveguide is a tapered optical waveguide having at least two different cross sections.

In embodiments the optical transmission (T0) of the waveguide is greater than 50%, for incoupled light having wavelengths between 180 nm and 25 µm, said optical waveguide having a length smaller than 2 m, preferably smaller than 0.5 m, more preferably smaller than 0.25 m. In variants ,optical transmission (T0), is greater than 80%, preferably greater than 90% for incoupled light having wavelengths between 300 nm and 5 µm, preferable between 350 nm and 2 µm, even more preferably between 400 nm and 700 nm.

In advantageous embodiments the optical waveguide is configured to be elastically stretchable up to at least 10%, preferably at least 20% , more preferably at least 30% of its length (L) and so that, after having been stretched, the optical transmission (T2) remains at least 90% of the transmission (T0) of the optical waveguide before being stretched.

The invention relates also to an optical waveguide bundle comprising at least three optical waveguides.

The invention relates also to a medical device comprising at least one optical waveguide of the invention. The medical device may be a cochlear implant. In another aspect the invention relates also to an optical sensor comprising at least one optical waveguide of the invention and an optical cavity sensor head arranged to said optical waveguide, the sensor head comprising an optical cavity closed by flexible membrane .

The invention is also achieved by a method of fabrication of an optical waveguide as described and comprises the steps (A-D) of:
A) realizing a hollow preform made of a thermoplastic elastomer (TPE)
B) reducing the diameter of said preform and elongating said preform until a capillary is formed having a predetermined length (L) and a predetermined cross section, said capillary having a central opening having a predetermined cross section;
C) introducing liquid silicone into the central opening of said preform;
D) polymerising said liquid silicone so as to form an optical waveguide having a core being made of polymerised liquid silicone;

In an embodiment step C and D are replaced by the steps E to G :
E) introducing liquid silicone during said step B of reducing the diameter of said preform;
F) while reducing the diameter of said preform keeping said liquid silicone in a liquid state until a predetermined length (L) and a predetermined cross section of a precursor optical waveguide is obtained;
G) thermal polymerising said liquid silicone and said capillary so as to form an optical waveguide.

In an embodiment B, C and D are replaced by the steps H to J :
H) after said step A, introducing a liquid polymer into the central aperture of said hollow preform;
I) reducing the diameter of the preform filled with liquid polymer and elongating said filled preform until a capillary is formed filled with liquid polymer , said capillary having a predetermined length (L) and a predetermined cross section;
J). polymerising said liquid polymer by applying UV light

In a variant said liquid polymer is liquid silicone or liquid siloxane.

### BRIEF DESCRIPTION OF THE DRAWINGS

FIG. 1 shows an optical waveguide of the invention;

FIG. 2 shows a longitudinal cross section of a waveguide of the invention illustrating its optical acceptance angle and the angle of an outcoupled light beam;

FIG. 3 shows a flat optical waveguide according to the invention;

FIG. 4 shows a tapered optical fiber of the invention;

FIG. 5 shows a lateral cross-section of a fiber bundle comprising optical fibers according to the invention;

FIGS. 6-9 show exemplary cross sections of different types of optical fibers having at least two light guide cores imbedded in a polymer cladding according to the invention;

FIG. 10 illustrates some steps of the fabrication of an optical waveguide of the invention;

FIG. 11 illustrates a hybrid preform that may be used to realize a Fan-In/Fan-out optical component according to the invention;

FIG. 12 illustrates a sensor head comprising an optical cavity that comprises an outer membrane, said cavity being arranged by an outer tube on an optical fiber of the invention;

FIG. 13 illustrates an optical fiber of the invention comprising a lateral incoupler grating and a lateral outcoupler grating;

FIG. 14 illustrated a cochlear implant comprising an optical fiber of the invention;

FIG. 15 shows an example of a portion of a preform comprising two holders to make a fan-in/fan-out optical component;

FIG. 16 shows a portion of a preform comprising two holders to make a fan-in/fan-out optical component, the two holders comprise a plurality of wires to be removed after injection of a TPE polymer.

FIG. 17 illustrates a preform after overmolding of a TPE polymer of the wires that are between the two holders of the arrangement of FIG. 16.

FIG. 18 illustrates the demolding of a hybrid multicore fiber and fiber bundle preform after the injection of a TPU layer as cladding layer;

FIG. 19 illustrates a multifiber Fan-in/Fan-out platform realized by using the mold of FIG. 16 and FIG. 17.

### DETAILED DESCRIPTION OF THE INVENTION

The present invention will be described with respect to embodiments and with reference to the appended drawings, but the invention is not limited thereto. The drawings described are only schematic and are non-limiting. In the drawings, the size of some of the elements may be exaggerated and not drawn on scale for illustrative purposes. The dimensions and the relative dimensions do not correspond to actual reductions to the practice of the invention.

As used herein, the term “optical waveguide” — defined also as waveguide — used herein encompass all types of homogeneous or non -homogeneous and/or tapered optical waveguides such as monomode and multimode fibers but also monomode and multimode flat optical waveguides and also waveguide bundles that comprise a plurality of optical fibers or flat optical waveguides or a mix of them. Waveguides 1 have a longitudinal axis which is defined as a central virtual axis of the waveguide 1 defined in the direction of the guidance of an optical light beam 100 in the waveguide 1. Optical guidance may be performed by total internal reflection (TIR) or by using reflecting or diffracting layers or structures. In the case of monomode waveguides only one mode is guided through the core of the waveguide. Said virtual axis defines a Z-axis and two orthogonal axes X, Y or directions to said Z-axis. Lateral cross sections herein are cross section defined in a X-Y plane. Longitudinal cross sections are defined in a plane comprising said Z-axis.

The types of the optical waveguide 1 of the invention will be chosen according to the type of application or geometric constraints and the geometrical and working temperature requirements of the aerosol-generating device wherein it is implemented and are typical, but not exclusively the following choices:
single fibers: for transmitting intensity, polarisation and spectral information;
fiber bundles: for transmitting images and illuminating light beams;
multi-core optical waveguides such as multi-core optical fibers
flat waveguides: for transmitting intensity, polarisation and spectral information, as well as the transmission of images and illumination light beams;
Fan-in/Fan-out optical devices.

Optical waveguides 1 such as optical fibers 1 and optical fiber bundles 300, such as used for example in endoscopes, are well known to the skilled person in the field of guided optics and are not describes further here. It is also known how to configure an optical fiber arrangement suited for illuminating an object and collecting reflected or transmitted light by such an object. The invention proposes optical waveguides such as optical fibers and a fabrication procedure of these fibers. As described further in detail, one of preferred fabrication methods includes a realisation of a thermoplastic elastomer (TPE), capillary preform 200, a such as thermoplastic polyurethane (TPU) preform 200, that may be thermally drawn to obtain a highly flexible TPU capillary 2000. Waveguides 1 of the invention are realized by either filling said preform 200 with a polymer and pulling them to obtain a thin waveguide 1, or by first realizing a solid capillary and filling it with a liquid polymer. Different ways of the curing of the liquid polymer of the core 10 are described further. The liquid to be used to form the core 10 of the waveguide 1 is preferably silicone, or siloxane

In a first aspect the invention proposes a new biocompatible and highly stretchable optical waveguides 1 by using low-cost, biocompatible and optically transparent materials with a much more reduced multimode behaviour. The general idea remains in the use of a thermoplastic polyurethane (TPE) for the cladding of the waveguide 1 , preferably thermoplastic polyurethane (TPU), capillary 2000 starting from a TPE preform 200. Afterwards, this liquid silicone could polymerize and create a final cladded optical fiber. The invention is not limited to waveguides having a solid core. The waveguide 1 may also be a hollow waveguide consisting solely of a capillary made of an elastomer. The capillary may have a coating of it internal surface.

It is understood that the waveguides 10 of the invention may be arranged in a wide variety of forms and geometries or may be arranged in any configuration in a medical device 2. In a first aspect the invention relates to an optical waveguide 1, comprising a core layer 10, defining a longitudinal axis Z, and a cladding layer 20 surrounding said core layer 10 , said optical waveguide 1 having an incoupling surface 31 for incoupling a light beam 110 into said core layer 10 and an outcoupling surface 51 for outcoupling light 120 out of said core layer 10, said core layer 10 and said cladding layer 20 being configured to transmit along said longitudinal axis Z a guided light beam 100, through said core layer 10 and from said incoupling surface 31 to said outcoupling surface 51, said guided light beam 100 having a wavelength greater than 180 nm. In all the embodiments of the invention the cladding layer of the optical waveguide 1 is made of a thermoplastic elastomer (TPE), that is also defined as thermoplastic rubbers. TPE’s are a class of copolymers or a physical mixture of polymers, usually a plastic and a rubber, that consists of materials with both thermoplastic and elastomeric properties. Thermoplastics components are relatively easy to manufacture, for example by injection moulding Thermoplastic materials present both the advantages of rubbery materials and plastic materials. The benefit of using thermoplastic elastomers in the waveguide of the invention is the ability to stretch to moderate elongations and return to its near original shape creating a longer life and better physical range than other material. The principal difference between thermoset elastomers and thermoplastic elastomers is the type of crosslinking bond in their structures. In fact, the crosslinking property is a critical structural factor which imparts the high elastic properties of the optical waveguide of the invention. TPE is well known and not further described here.

There are six generic classes of commercial TPEs (designations according to the ISO norm 18064):
styrenic block copolymers, TPS (TPE-s)
thermoplastic polyolefinelastomers, TPO (TPE-o)
thermoplastic Vulcanizates, TPV (TPE-v or TPV)
thermoplastic polyurethanes, TPU (TPU)
thermoplastic copolyester, TPC (TPE-E)
thermoplastic polyamides, TPA (TPE-A)
not classified thermoplastic elastomers, TPZ

Herein, TPU is a preferred TPE but other TPE materials may be used for the cladding layer.

The core layer 10 is made of a first material having a first index of refraction n1, and the cladding layer 20 is made of at least one layer of TPE having a second index of refraction n2 that is smaller than said first index of refraction n1 In a specific case of a capillary made of at least one TPE layer that comprises an internal reflection layer, the core layer 10 may be air or vacuum or may be a liquid.

The values of refractive indexes can cover a broad range for both materials for example n2= 1.49 - 1.57 and n1 = 1.52 - 1.60.

The limitation of the useful length of the proposed optical waveguide 1 is the penetration length of the liquid silicone into the TPU capillary as further described in the method section. Different fabrication methods are possible that are described further, and each method provides to achieve different geometries and different lengths in function of the application and so the required intensity throughput. Nevertheless, a typical optical waveguide is an optical fiber having a fiber length of around 10 cm, for example in the case of medical implants, but it may longer. For example, a length of 10 cm would be enough for an organ-scale distance that is over 10 cm for humans [Ref. 5].

In preferred embodiments, the lineal losses of the optical waveguide do not exceed 0.5 - 1 dB/cm, which ensures an overall loss of maximum 10-20 dB for a round trip on a 10 cm long waveguide. The bending losses are of high importance in applications as cochlear implants [3] where the waveguide must be arranged on tight radii as we can found inside a cochlea (1-2 mm at the far end). However, these radii are progressive and the waveguide 1 may be placed through places having very short bending radii only over a few millimetres. In order to provide some margin on the final retrieved signal coming from a resonating cavity at a fiber tip for example, it is essential that the waveguide has less than 5 dB loss on a round trip base for a bending radius of about 5 mm over a fiber length of 30 mm that corresponds to an average cochlear length. Obviously, this constraint is driven by the fiber attenuation itself. If the fiber attenuation is much lower than the 0.5 - 1 dB/cm specified above, then the margin available for the bending loss will be higher.

Typical attenuation values are 0.79 dB/cm at 1550 nm and 0.46 dB/cm at 1300 nm. At 633 nm, attenuation values are lower 0.79 dB/cm at 1550 nm and 0.46 dB/cm at 1300 nm So, fiber lengths of more than 2 m may be used. Experimental data have shown that the optical waveguide of the invention has a lower attenuation in the visible part of the spectrum than in the infrared part of the spectrum.

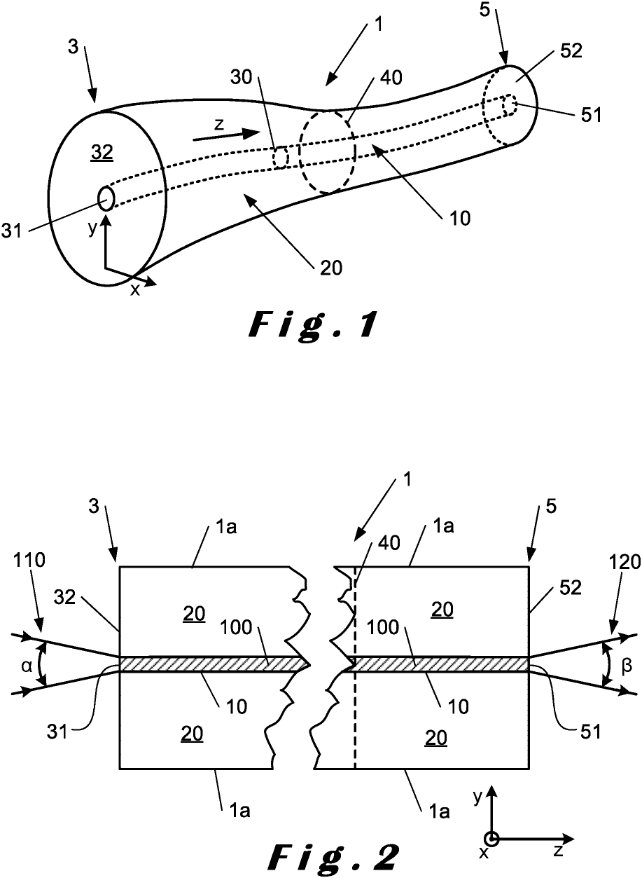

In a preferred embodiment the optical waveguide 1 an optical fiber that may be a monomode or a multimode fiber, as illustrated in FIG. 1. The lateral cross section 30 of the core 10 and the cross section 40 of the cladding may be uniform over the length of the waveguide 1, but may also vary as illustrated in FIG. 4

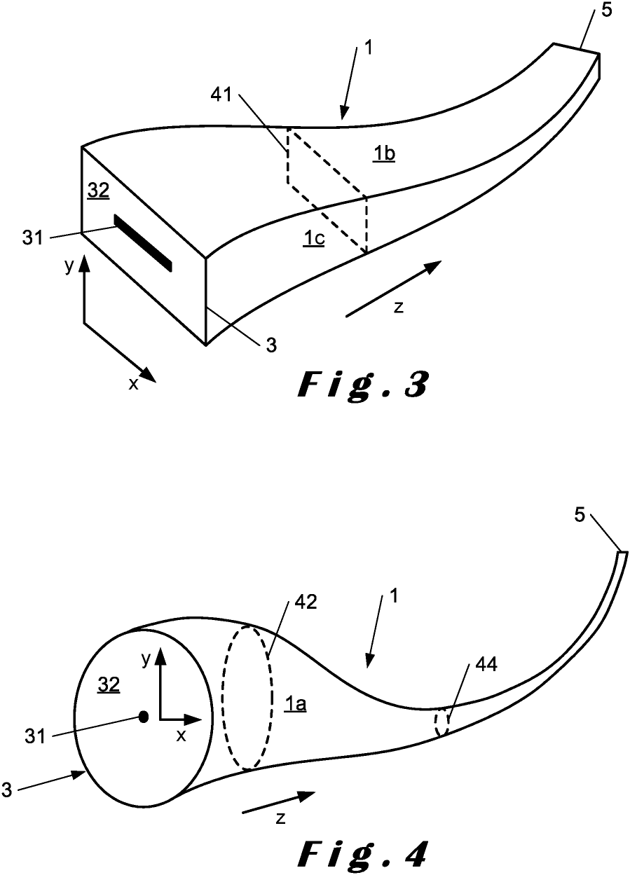

In an embodiment, illustrated in FIG. 3 the optical waveguide 1 has a first lateral side 1c having a first width W1 and a second side 1b having a second width W2 that is larger than said first width W1, said widths W1, W2 being defined in any lateral cross section, defined in an plane X-Y orthogonal to said longitudinal axis Z. FIG. 3 shows a flat optical waveguide 1 having a rectangular shaped cross section, but other cross sections may also be possible, such as elliptical shaped cross sections, or trapezium shaped cross-sections.

In an embodiment, illustrated in FIG. 4, the optical waveguide 1 is a tapered waveguide 1, having a tapered form in at least one plane comprising said longitudinal axis Z. FIG. 4 illustrates a varying shape and/or dimension of lateral cross sections 42, 44

In an embodiment said core layer 10 is made of a polymer. This polymer may be silicone.

In variants the core layer may be a liquid. This may be realized by providing a capillary that has a very small core diameter so that the liquid remains trapped inside the optical waveguide 1. In a variant the input and output areas of a waveguide having a liquid core may have a window to close of the liquid core so that the liquid remains inside the waveguide 1

In an embodiment said cladding layer 20 is made of polyurethane, possibly a thermoplastic polyurethane (TPU).

In an embodiment a reflecting layer may arranged between said core layer 10 and said cladding layer 20, said reflecting layer being arranged to provide inside said core layer 10, total reflection and guidance of incoupled guided light into said core layer 20. Said reflecting layer may be a metallic layer or a dielectric layer or a combination of them.

In an embodiment the optical waveguide 1 is an optical fiber wherein said core layer 10 and said cladding layer 20 are configured to guide a number of modes less than 100, preferably less than 20, more preferably less than 5.

In an embodiment the optical waveguide 1 is a monomode fiber

In an embodiment the optical waveguide 1 is configured to guide less than 100 modes, preferably less than 20 modes, more preferably less than 5 modes, defined in at least one longitudinal plane X-Z, Y-Z.

In an embodiment the optical waveguide is a tapered optical waveguide having at least two different cross sections 42, 44.

The optical waveguide 1 has an optical transmission T0, defined as the ratio I2/I1 of the intensity I2 of the outcoupled light 120 to the intensity I1. In an embodiment the optical waveguide 1 has a practical length smaller than 2 m, preferably smaller than 0.5 m, more preferably smaller than 0.25 m. and the intensity I2 of the outcoupled light 120 may be greater than 10%, preferably greater than 30% than the intensity T0 of the incoupled light 110, for incoupled light having wavelengths between 180 nm and 25 µm.

In an embodiment a useful length of the optical waveguide 1 has an optical transmission that is greater than 80%, preferably greater than 90% for incoupled light having wavelengths between 300 nm and 5 µm, preferable between 350 nm and 2 µm, even more preferably between 400 nm and 700 nm.

In the case of medical implants for example. said useful length is typically 10-20 cm.

In an embodiment the optical waveguide 1 can be elastically stretched up to at least 10% of its length L and so that, after having been stretched, the optical transmission T2 remains at least 50%, preferably at least 70%, even more preferable at least 90% of the transmission T0 of the optical waveguide 1 before being stretched.

One of the essential features of the optical waveguide 1 is that it may be stretched while maintaining substantially its optical guidance properties. In the case of a hollow core waveguide 1 made of TPU the core 10 is air or vacuum, and an elongation of 600% is possible before breakage of the waveguide 1. In the case of an optical waveguide 1 having a core 10 made of silicone the possible elongation before rupture may be similar depending on the adherence properties of the core layer 10 with the cladding layer 20. The rupture limit may also depend on the elongation properties of the core layer because, depending on the chosen core material layer, the core layer may be damaged or ruptured before the damaging of the cladding layer. Typical silicone core layers may have an elongation of up to 50% before rupture.

In an embodiment an adherence or an antifriction layer may be provided at the inner surface of said capillary 2000 before introducing said liquid core material. This provides ways to improve the breakage limit or possible mechanical damages to the waveguide 1 for example in situations of small curvature radii and/or high traction forces.

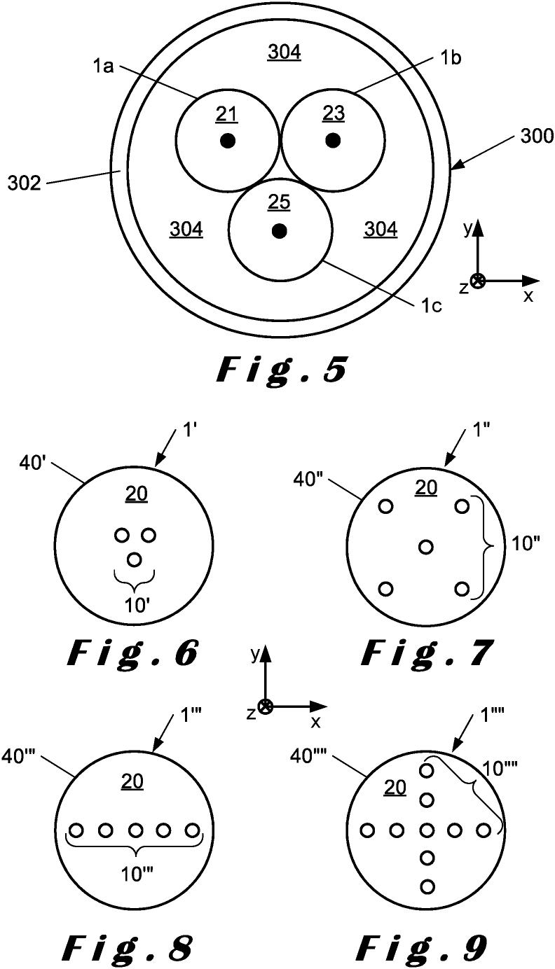

The invention is also achieved by an optical waveguide bundle 300, illustrated in FIG. 5 comprising at least three optical waveguides 1a, 1b, 1c as described. In a variant 7 optical fibers 1 may be arranged into such a fiber bundle 300 that comprises an outer mantle 302 and an inner filling material 304.

In embodiments illustrated in FIGS. 6-9 an optical waveguide 1′, 1″, 1‴, 1⁗ may comprise a plurality of core layers 10′, 10″, 10‴, 10⁗.

In embodiments the optical waveguide (1) may be a polarization maintaining waveguide (1).

It is understood that the optical waveguide 1 of the invention is not limited to only a waveguide 1 comprising a core layer 10 and a cladding layer 20. As well the core layer 10 and/or the cladding layer 20 may comprise structured portions that have an optical function. A typical optical structure is a diffraction grating that may be a local diffraction grating or a distributed grating, as illustrated in FIG. 13. Also, hologram-type structures or layers may be arranged into or on said optical waveguide 1.

In advantageous embodiments, at least a portion of said waveguide 1 is arranged according to a resonant waveguide grating (RWG). RWG’s are described in for example:
A.Sharon et al.:“Resonating grating-waveguide structures for visible and near-infrared radiation”: J.Opt.Soc.Am″ vol. 14, nr. 11, pp.2985-2993, 1997

RWG’s are made by using a multilayer configuration and combine subwavelength gratings and a thin waveguide. A resonance occurs when incident light is diffracted by a grating and matches a mode of the waveguide. As most of the spectrum of incoupled light does not couple into the waveguide, strong spectral effects are provided in reflection and/or transmission. This to the fact that RWG’s are corrugated waveguides and behave as a waveguide-grating. The use of RWG in indicia allows to provide unique optical effects that are extremely difficult to identify and to duplicate. RWG’s are generally designed to have spatial periodicity shorter than the wavelength they operate with and are therefore called “subwavelength” structures or subwavelength devices. Eventually they have periodicities closed to the wavelength they are operating with and just above it. Quite often, the periods are significantly smaller than the free-space wavelength they are working with, for example a third of it. Because of their small periodicity, they do not allow various diffractive orders, which distinguishes them from much simpler diffractive optical elements (DOE).

Using RWG allows to provide unique incoupling and outcoupling optical effects, for example by providing a high incoupling and/or outcoupling efficiency or to incouple and outcouple polarized light beams more efficiently or with predetermined angles which would not be possible by using ordinary diffraction gratings such as binary diffraction gratings. RWG may be realized by embossing techniques allowing to provide cheap waveguide that have very efficient light coupling efficiencies that may depend, according to their design, particularly on specific predetermined wavelengths. In variants that are not illustrated in figures at least one of the lateral surfaces of the waveguide 1 is arranged, continuously or discontinuously, over at least 50% of its entire length, as an incoupling surface and/or an outcoupling surface. Said incoupling surface and/or an outcoupling surface may be configured as a RWG.

The invention is also achieved by optical systems or sensors comprising at least one optical waveguide 1 as described herein. In an example, a resonating cavity is arranged as a tip of the optical waveguide 1 of the invention. As a light source low-cost telecom-grade LEDs may be used to interrogate the resonating cavity. In such devices a useful fringe visibility is required in cavity lengths of around 200 - 300 µm.

The invention is also achieved by an optical waveguide sensor that comprises a resonating cavity 520 arranged in a tip fixed to the output end of the optical waveguide deformable diaphragm 530. The cavity 520 may have another function than a resonating effect, for example the cavity may provide, through the deformation of the membrane, a varying light intensity of the lightbeam that is sent back into the fiber 1. In embodiments cavities 520 may be filled with air or a liquid , such as oil.

In embodiments the medical device is an implant to be used in cohleas. FIG. 14 illustrates a cochlea implant 600 that comprises a central portion 606 to be inserted into the ear of a human being. A mechanical guidance structure 606 is arranged to said central portion and comprises at least one optical waveguide 1 according to the invention.

In advantageous embodiments the medical device may comprise at least one optical waveguide 1 of the invention to provide a UV-light beam to a predetermined location, for example to disinfect a location in a living body. It is generally understood that at least one extremity of the optical waveguide 1 may have a shape so that it may be used for an optical function such as the deviation or focusing or diverging of an incoming or outcoupled light beam. Said shape may be realized during the fabrication process of the waveguide 1, for example by heating the extremity so that a rounded shape is provided to an end of the waveguide.

The waveguide 1 of the invention may be used for optogenetics described in Ref.18. The inventio. is related also to a device to be used in optogenetics and that comprises at least one waveguide 1 according to the invention.

The waveguide 1 of the invention may also be used to track in real-time surgical instruments, for example to give information of the localisation of the tips of the instruments or to monitor optical information at the tip of the optical waveguide at the place of a surgical intervention. The invention is therefor also related to a surgical instrument that comprises the optical waveguide of the invention.

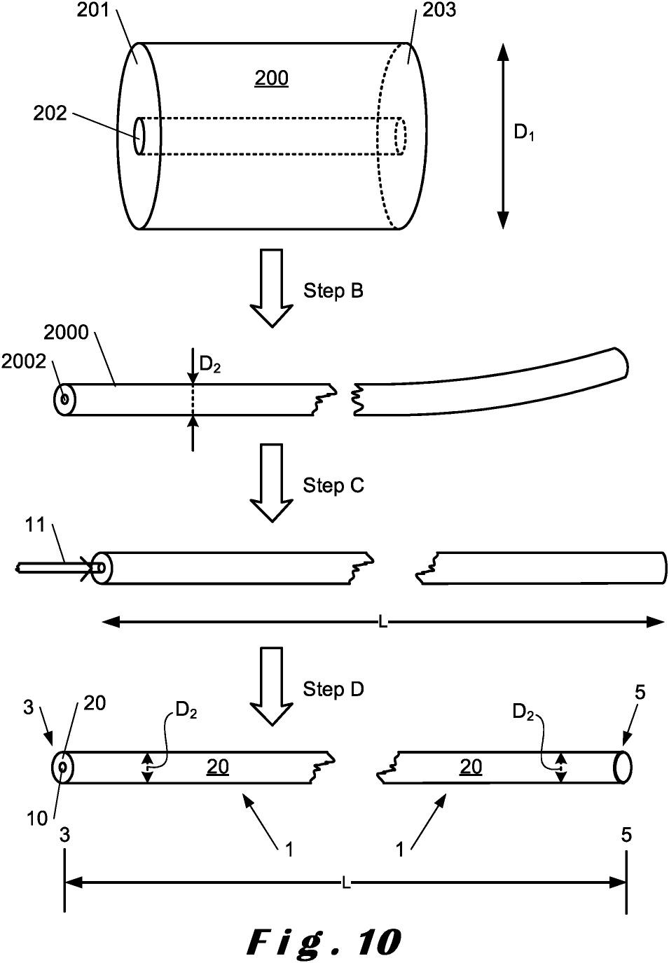

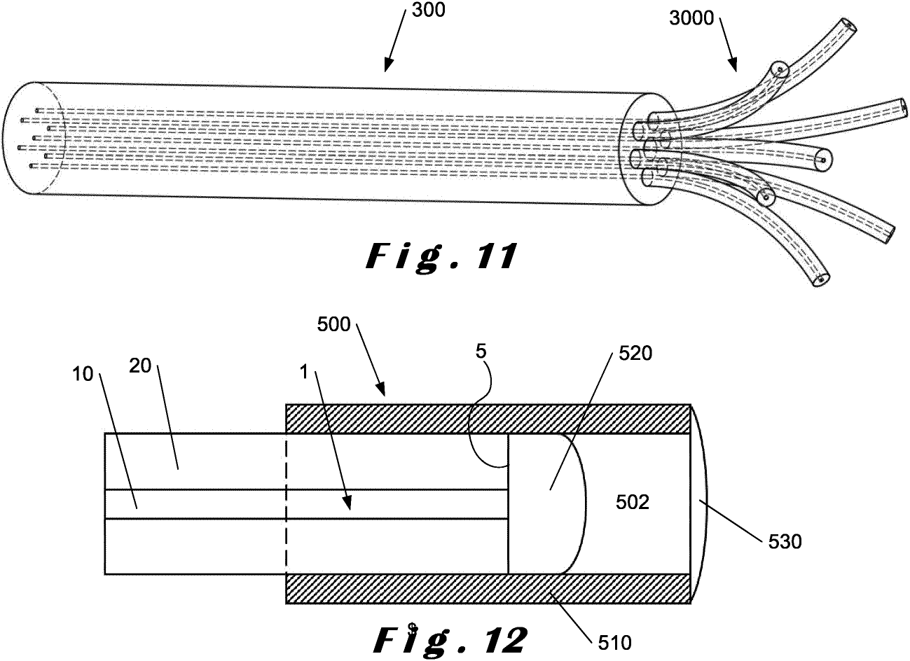

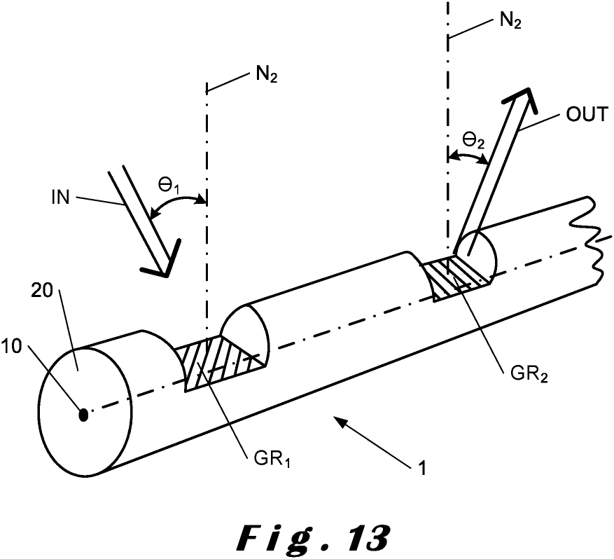

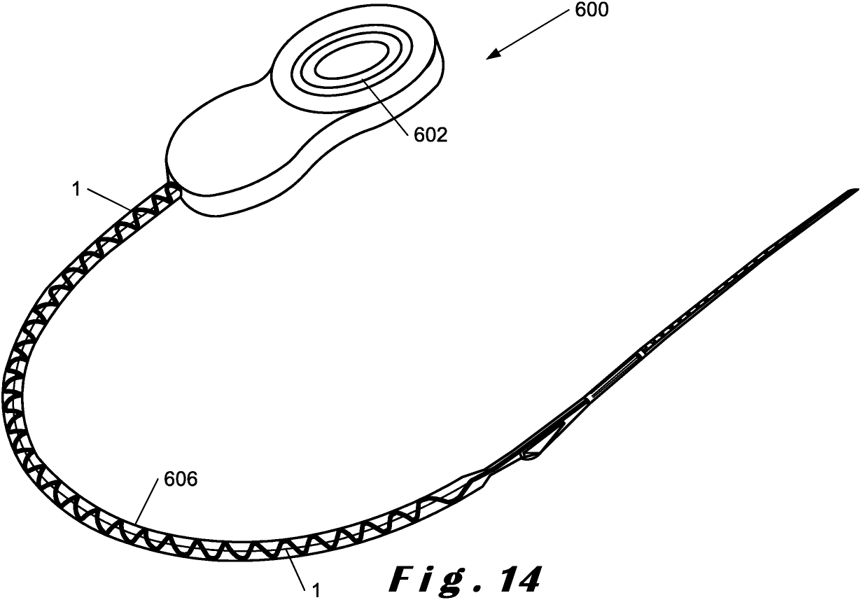

In a second aspect the invention relates to the fabrication of an optical waveguide 1 as described before, and comprises the steps (A-D) That are illustrated schematically in FIG. 10:
A) realizing a hollow preform 200 made of a thermoplastic elastomer (TPE), preferably a thermoplastic polyurethane (TPU);
B) reducing the diameter of said preform 200 and elongating said preform until a capillary 2000 is formed having a predetermined length L and a predetermined cross section 40, 41, said capillary having a central opening 220 having a predetermined cross section 30
C) introducing liquid silicone 11 into the central opening 220 of said preform;
D) polymerising said liquid silicone so as to form an optical waveguide 1 having a core 10 being made of polymerised liquid silicone 11 and having a predetermined length L and an outside diameter D2. The diameter of the core is directly related to the proportion of the outside diameter of the preform D1 and the diameter of the aperture of the preform, because this proportion does not change during the diameter reduction step B;

In an embodiment step C and D are replaced by the steps E, F, G :
E) introducing liquid silicone 11 during said step B of reducing the diameter of said preform;
F) while reducing the diameter of said preform 200 keeping said liquid silicone in a liquid state until a predetermined length (L) and a predetermined cross section 40, 41 of a precursor optical waveguide is obtained;
G) thermal polymerising said liquid silicone 11 and said capillary (2000) so as to form an optical waveguide 1.

In an embodiment steps B, C and D are replaced by the steps H, I, J:
H) after said step A, introducing a liquid polymer 11 into the central aperture 202)of said hollow preform 200)
I) reducing the diameter of the preform 200 filled with liquid polymer 11 and elongating said filled preform 200 until a capillary 2000 is formed filled with liquid polymer 11 , said capillary having a predetermined length L and a predetermined cross section 40, 41;
J). polymerising said liquid polymer by applying UV light

In an embodiment step said liquid polymer is liquid silicone, possibly a liquid siloxane

In an embodiment of the method said obtained optical waveguide 1 is a multimode optical fiber. In a variant, said obtained waveguide is a mono-mode optical fiber.

In an embodiment said obtained waveguide is an optical waveguide having a non-circular cross section defined in any lateral plane X-Y. In embodiments the core 10 of the waveguide has a very small cross section, which may be smaller than 1 µm. A process to realize this is now described

In order to decrease the fiber diameter during the drawing, we will have to adjust the drawing parameters as the winding rate that will reduce the fiber diameter. In order to do so, both materials, TPU and silicone must be able to be processed at such small geometries. For silicone, that will be quite easy as it will remain liquid and will follow the TPU deformations. If the final fiber diameters are not small enough, it will be possible to start a new drawing process, if silicone has not yet polymerized, in order to reduce the fiber size. This process would be similar as the one proposed in Ref. 16 wherein an introduced fiber is melted while a rotating rod coils the microfiber. In our case the melting would be performed by the same heaters that are used to draw the fiber from the preform. Due to the small size of the incoming fiber, not greater than 100 µm, the melting should be achieved rapidly and the silicone polymerization won’t have the time again to happen. Coiling around a rod is mandatory rather than around a normal spool. The reason is that warm TPU fibers will get stuck to each other if they are not cooled enough. For a normal optical fiber drawing, this cooling is performed by taking away the furnace and the winding spool. This cannot be the case for the micro and nano fibers where the winding process must be done as close as possible to the furnace in order to avoid a probable fiber break. Also, if the micro and nano fibers enter in contact, they will also most probably get stuck and due to their small size, a separation would be impossible. So, winding around a rod and separating the fibers at each turn, by longitudinally moving the rod, is the only solution.

In an embodiment a length of useful optical waveguide is determined by cutting the waveguide 1 until an acceptable optical transmission is obtained. So, a transmission measurement step may be performed wherein the optical transmission ratio T1/T0 of said short optical waveguide is determined followed by a new step F consisting in cutting another predetermined length of said optical waveguide 1, so as to provide a second short optical waveguide having a length smaller than the length of said first short optical waveguide, said second short optical waveguide having a higher transmission ratio T2/T1 than the transmission ratio T1/T0 of said first short optical waveguide.

In variants, micro or nano-sized optical fibers may be realized. Their diameters may be typically 1 µm, possibly less than 1 µm. In case a preform made of TPU is filled with UV polymerizable glue or silicone, the liquid that is introduced in the central aperture will follow the deformation of the TPU cylinder during its reduction of diameter so that the central aperture 2002 is not closed during the capillary pulling operation even when micro-sized diameters are reached. Once the predetermined length and outer diameter is reached the internal liquid is polymerized by UV light, through said TPE or TPU.

The invention relates also to a method of fabrication of a system that may be used to realize a Fan-IN/OUT optical system (Ref.17), allowing to connect the outputs and inputs of a multicore fiber (Ref.17), This method comprises preferably the steps of:
providing a plurality of optical fibers having a non-cured liquid core and having a diameter typically of 40 µm;
aligning the plurality of fibers in a mechanical holder, to form a bundle, having a plurality of holes and the same fibers geometry distribution as the multicore fiber. The mechanical holder has preferably a cylindrical external diameter that is equal or greater than the multicore fiber to connect.
in an embodiment , the number of fibers introduced in the holder is 7. The introduced fibers may be different types of fiber;
gluing the assembly of the mechanical holder, preferably by a UV glue, in order to fix all components together.

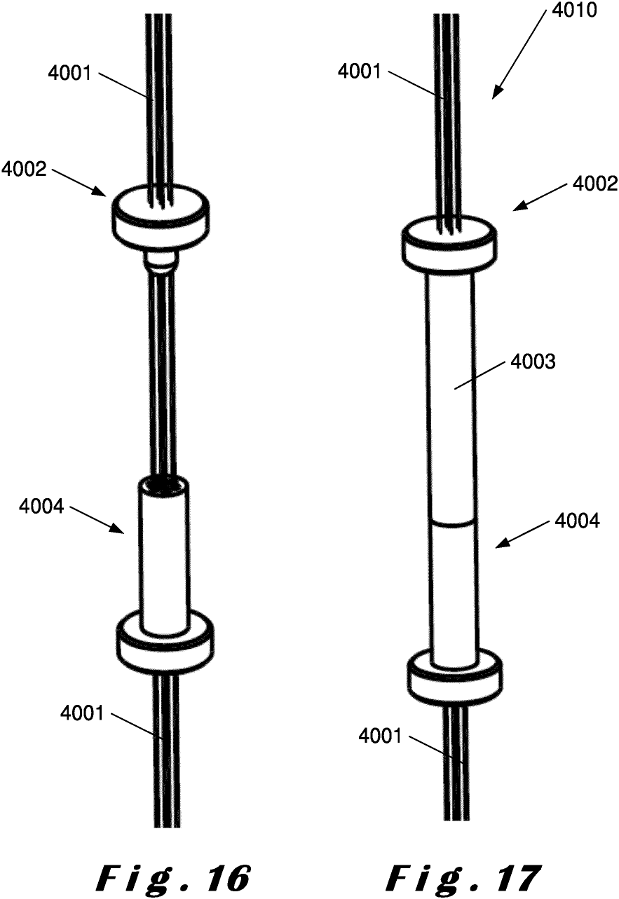

In a variant, illustrated in FIG. 17, the preform is a hybrid preform. For example, a block 4000 may comprise two holders in which wires are arranged (as illustrated in FIG. 16) and this structure may be over moulded by a TPE, such as a TPU, cladding layer as illustrated in FIG. 17. The preform may be produced by injection moulding and the first half of the preform constitutes a multicore preform. The second half is a plurality of preferably 7 independent tubes directly aligned and connected to the first part of the preform comprising said two holders.

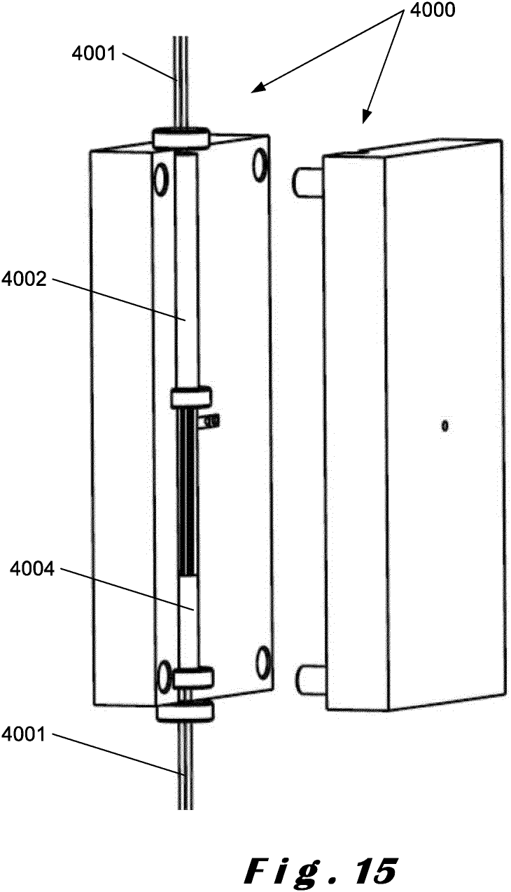

FIG. 15 shows an example of an arrangement 4000 comprising a portion of a preform comprising two holders 4004, 4004 , arranged preferably to realize a fan-in/fan-out optical component.

FIG. 16 shows a portion of a preform comprising two holders of FIG. 15, to make a fan-in/fan-out optical component, the two holders comprise a plurality of wires 4001 to be removed after injection of a TPE polymer FIG. 16 illustrates two inserts comprising 7 mold cores before injection of the cladding; After over molding the volume between the two holders 4002, 4004 and so the wires present in that volume (FIG. 16) by a TPE layer 4003. The TPE is then cooled and the wires 4001 are withdrawn, leaving a plurality of axial holes in which another polymer, such as silicone may be introduced. The so formed preform may be drawn to make either a multicore fiber or may be drawn or heated over its central part so as to form a multi-core structure having a central portion with a reduced diameter, which may be used in a Fan-in/Fan-out optical device.

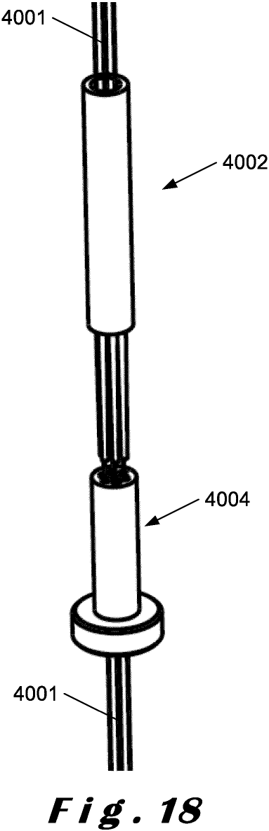

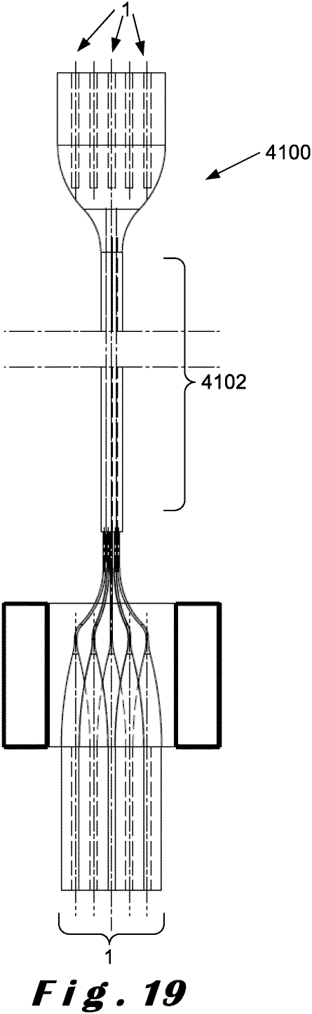

FIG. 18 illustrates the demolding process of such a multicore fiber after the injection of a TPU layer 4003 as cladding layer illustrated in FIG. 17.FIG. 19 illustrates a multicore Fan-in/Fan-out platform realized with the mold of FIG. 16 and FIG. 17, and after melting and drawing to reach the final desired diameters. The fan-in/fan-out component 4100 illustrated in FIG. 19 comprises two ends 4104, 4106 having spaced cores and a middle section 4102 wherein the cores are closer than at the two ends 4104 and 4106.

The melt and draw process is realized in the transition area of the hybrid preform in order to decrease the size of the multicore preform part to a normal fiber size and a lower to the normal size for the tubes outgoing from the transition area.

In an embodiment the fibers may be arranged in a ring or tube forming a multicore fiber structure-like. This structure is fixed by a thermal flash and then thermally drawn to reach a final diameter equal to the multicore fiber to be connected to.

The invention relates also to an illumination device and method of illumination that is based on the use of varying diameter of the core of a fiber. Such an illumination method comprises the steps of:
providing an optical waveguide 1 as described herein;
introducing light into the core 10 of the optical waveguide 1
stretching portions of the optical waveguide 1 so as to couple light out of the core 10 to its side and through said cladding 20.

In a variant the stretching may be made periodically stretched by an automated mechanism. In an embodiment, dopants may be integrated in the TPE cladding layer to provide light diffusion effects by the cladding layer. By integrating dopants it is possible to make more visible stretched portions of the optical waveguide and so provide lighting effects, useful in for example light decorations.

### Experimental Results

In a typical process of a TPU capillary 2000, the first step is to produce a cladding preform 200, preferably by injection moulding. The cladding dimensions has to respect the ratio of the final cladding diameter and core fiber diameter in order to obtain a multimode fiber having a predetermined number of guides modes, or to ensure a fiber and core size that may be close to or equal to standard single mode fibers. The cladding diameter must comply with the moulding dimension constrains but also depends on the initial central hole diameter that is needed to use an insert inside the mould. This insert is a critical component of the cladding production as it can be crooked by the hot polymer flow during the moulding process. Based on these constraints and typical injection moulding equipment, typical cladding diameters are up to 20 mm for a length of about 100 mm. The central hole diameter of the preform 200 is normally not less than 1 mm because of the polymer flow stress during moulding. Hole diameters of the preform as low as 0.5 mm may be obtained. Another constraint on the insert is its surface quality and adherence to the TPU. The surface quality will directly influence the optical interface between TPU and silicone, and fortiori, the optical guidance losses inside the final fiber. Thus, a surface treatment is in principle needed in order to reduce the roughness and the TPU adherence. Once the cladding preform is obtained, a standard process of thermal fiber drawing provides a hair-thin TPU fiber capillary. Because the lengths of fiber that are needed for most applications addressed here, are quite short (10-15 cm), the conicity and diameter fluctuations are less constricting and hence relax the process constraints.

Tests have been performed to realize silicone penetration in small diameter capillaries, glass capillaries 2000 are used having a central hole 2002 of 15 µm and external diameter of 125 µm. By using the syringe technique, it is possible to inject silicone in a length of around 100 mm. By using vacuum pump techniques together with a syringe, it is possible to achieve a penetration depth of more than 100 mm. By modifying the wettability of the inner capillary walls, hence with the help of the capillary filling force, the initial penetration length is improved. In addition, by using more performant vacuum pumps, and by increasing the syringe efficiency on the other side of the fiber, penetrations depths over fiber lengths up to 20-40 mm are achievable.

A realized optical fiber 1 according to the invention has the following properties.

The core material is made of a silicone that is usually used as a LED liquid encapsulant. The silicone is an OPTOLINQ trademark OLS-5291-type silicone commercialized by Caplinq Corporation (Canada). The achieved core dimension was:

The cladding material was a TPU polymer that can be found at very soft grades, such as the BASF 1185A TPU. The achieved cladding dimension was 200 µm and the core diameter 50 µm and the length of the fiber 1 was 250 mm.The fiber had a high numerical aperture of 0.32 It has been possible to stretch the realized fiber 1 by about 50%, i.e. elongating the optical fiber up to 375 mm without notable optical losses. Bending losses were less than 20-30% due to the high numerical aperture of the fiber1. This cannot be achieved by other polymer-based fibers of prior art such as PMMA fibers.

Also, tests have shown that squeezing the optical fiber 1 of the invention does not alter its mechanical or optical properties. For example, by applying a lateral force of 20N -30N the optical fiber returned to its initial shape without any changes of its mechanical or optical properties.

Optical transmissions of the optical waveguide 1 have been measured by an optical bench.

At 633 nm the attenuations were 0.2 - 0.3 dB/cm. At 1300 nm the attenuation was: 0.3 - 0.5 dB/cm and at 1550 nm a typical attenuation was 0.6 - 0.9 dB/cm.

The optical waveguide of the invention may be used for some UV applications, if the lengths are typically shorter than 100 mm. In the UV, estimates of transmission were about 30-60% at 300 nm for a length of 50 mm, but the transmission value may vary considerably according to the type of polymers that are used and of course of the UV wavelength, Transmissions below 300 nm are typically lower than 20-10% for lengths of fiber of about 50 mm..

### Applications of the Waveguide 1 of the Invention

One of the important applications of the optical waveguide of the invention 1 relates to implants and more specifically cochlear implants. The optical waveguide 1 has been implemented in a pressure sensor tip arranged at a cochlear implant tip. The waveguide 1 is intended to minimize accidental structural intracochlear damage. Recent studies have demonstrated pressure pulses equivalent to sound levels causing severe impulse trauma during implantation, caused by the insertion of the electrode array into the enclosed space of the cochlea. A solution implementing the optical waveguide 1 of the invention will improve surgery reliability and flexibility and also the implant quality by avoiding any failure during the critical process of implantation. A cochlear implant cost, including surgery, can vary between USD 30′000 - 50′000. A failed surgery, though rare, will induce a second surgery preceded by a patient recovery time whilst an implant dysfunction (due to structural damage) is potentially possible as well. Additionally, the residual structures and neural tissue in the cochlea are highly relevant to the sound quality experienced by the patient. The insertion process can be highly traumatic to these structures, due to pressure pulses caused by surgeon handling and penetration of important membranes. Novel optical fiber technologies may revolutionize the surgical procedure, reducing fibrotic tissue growth and also maintaining the cochlear condition for successful future regenerative therapies to enhance the outcome. Furthermore, implantable optical technology could also improve the post-operative rehabilitation by continuously monitoring physiological parameters. The optical waveguide of the invention allows to decrease dramatically implants failures as well as surgery failures by providing a feedback measurement of important physiological or environmental parameters. Furthermore, it opens the door for long term and highly localized monitoring of physiological parameters. This is of high interest as it will enable to make an early detection of medical issues, enabling in this way to minimize possible post-implantation traumas and their consequent surgeries. It is also providing a solution to diminish wastes by increasing the reliability of implants and the consumables needed for surgeries. Subsequently, the potential diminution of wastes is obviously enhancing the energy efficiency of the implantation activities by reducing the implantation failures and their possible traumas but as well by preventing important surgeries on a long-term point of view.

Other important applications that may profit from the optical waveguide 1 of the invention are, but not exclusively:
ophthalmology: a pressure sensing device using the optical waveguide 1 is useful in cataract surgery;
spinal traumatology: Pressure monitoring after surgery (vertebra or disc replacement);
heart traumatology: blood flow and pressure monitoring after a heart attack, localized micro-surgery;
cardiac surgery, orthopedics, urology, and neurology;
security and counterfeit applications;
industrial sensor applications.

As a totally new class of optical waveguide, different applications of the waveguide of the invention could generate new markets. As an example, Bragg gratings may be provided on the waveguide 1 to improve its sensitivity with no need of any additional sensor. The waveguide 1 of the invention can be used for sensing, as explained for implants manufacturers, but could also be used to bring light at remote locations where glass optical fibers would be hazardous to use. The optical waveguides 1 of the invention may also be used in FAN/IN-FAN/OUT systems, such as for example described in Ref.15.

Still other applications address decorative illumination devices. A TPE such as TPU cladding may be doped with diffusing particles. In an example, light may be made more or less visible in sections where the core has a reduced size by pulling a section of the optical waveguide. In other applications Bragg gratings may be arranged on the optical waveguide 1 of the invention and allow to replace for example SiO2-based optical waveguides or fibers. Such waveguides 1 may be used for cryogenic applications where the thermal dilation of SiO2 is substantially non existing.

### Back-Ground Information and References

Gerd Keiser, Fei Xiong, Ying Cui and Perry Ping Shum, “Review of diverse optical fibers used in biomedical research and clinical practice”, Journal of Biomedical Optics 19(8), 080902 (August 2014)
Sedat Nizamoglu, Malte C. Gather, and Seok Hyun Yun, “All-Biomaterial Laser Using Vitamin and Biopolymers”, Adv. Mater. 2013, 25, 5943-5947
M. Llera, T. Aellen, J. Hervas, Y. Salvade, P. Senn, S. Le Floch and H. Keppner, “Liquid-air based Fabry-Perot cavity on fiber tip sensor”, Opt. Express 24, 8054-8065 (2016)
Sara T. Parker, and al., “Biocompatible Silk Printed Optical Waveguides”, Adv. Mater.2009,21,2411-2415
Myunghwan Choi, Matjaž Humar, Seonghoon Kim, and Seok-Hyun Yun, “Step-Index Optical Fiber Made of Biocompatible Hydrogels”, Adv. Mater. 2015, 27, 4081-4086;
M. Han, and A. Wang, “Exact analysis of low-finesse multimode fiber extrinsic Fabry-Perot interferometers”, Applied Optics (43), pp. 4659-4666, 2004;
Brandon W. Swatowski, Chad M. Amb, W. Ken Weidner, Ranjith S. John, Jeffrey D. Mitchell, “Advances in manufacturing of optical silicone waveguides for high performance computing” 2014 IEEE Avionics, Fiber-Optics and Photonics Technology Conference (AVFOP), ThB1, 2014
Md Rejvi Kaysir, Alessio Stefani, Richard Lwin, and Simon Fleming, “Flexible optical fiber sensor based on polyurethane”, 2017 Conference on Lasers and Electro-Optics Pacific Rim (CLEO-PR), 2017
Timo Stöver and Thomas Lenarz, “Biomaterials in cochlear implants”, GMS Curr Top Otorhinolaryngol Head Neck Surg, 2009
http://info.hotims.com/45604-161
Hugh L. Narcisco, Jr., “Silicone optical waveguide”, US5237638A, 1991
Nathan N. Haese, and al. “Pressure sensor utilizing a polyurethane optical fiber”, US4915473A, 1989
https://www.aliexpress.com/
Herve Elettro. Elastocapillary windlass: from spider silk to smart actuators. Mechanics of the fluids [physics.class-ph]. UPMC, 2015.
O.Shimakawa et al. Connector type fan-out device for multi-core fiber , SEI Tech Review nr 77, pp.23-28, Oct.2013.
[M. Sumetsky, Y. Dulashko, S. Ghalmi, “Fabrication of miniature optical fiber and microfiber coils”, Opt. Lasers Eng., 48 (2010), pp. 272-275].
O. Shimakawa, H. Arao, M. Shiozaki, T. Sano, A. Inoue, “Connector type fan-out device for multi-core fiber”,SEI Tech. Review, no. 77, pp. 23-28, October 2013.
S.Chen et al., “Near-infrared deep brain stimulation via upconversion nano-particle-mediated optogenetics”, Science 359, 2088, pp.679-684, February 2018.

1-29. (canceled)

30. An optical waveguide, comprising a core layer, defining a longitudinal axis Z, and a cladding layer surrounding said core layer , said core layer and said cladding layer being configured to transmit along said longitudinal axis Z a light beam having a wavelength greater than 180 nm. wherein
- said core layer is made of a first material having a first index of refraction,
- said cladding layer is made of at least one layer made of a thermoplastic elastomer having a second index of refraction being smaller than said first index of refraction.

31. The optical waveguide according to claim 30, wherein said at least one layer of thermoplastic is one of: a styrenic block copolymer, thermoplastic polyolefinelastomers, thermoplastic Vulcanizate, thermoplastic polyurethanes, thermoplastic copolyester, thermoplastic polyamides or not classified thermoplastic elastomers,.

32. The optical waveguide according to claim 30 being an optical fiber.

33. The optical waveguide according to claim 31, wherein said optical fiber is a monomode optical fiber.

34. The optical waveguide according to claim 30 having a first lateral side having a first width and a second side having a second width larger than said first width, said widths being defined in any lateral cross section, defined in an plane orthogonal to said longitudinal axis Z.

35. The optical waveguide according to claim 30, wherein said core layer is made of a polymer.

36. The optical waveguide according to claim 35, wherein said core layer is made of silicone.

37. The optical waveguide according to claim 30, wherein said core layer has an index of refraction of 1 and wherein the inner surface of said cladding layer 20 comprises a metallic and/or dielectric layer arranged to reflect light that is incoupled into said core layer.

38. The optical waveguide according to claim 34, being configured to guide less than 100 modes, defined in at least one longitudinal plane.

39. The optical waveguide according to claim 30 being a tapered optical waveguide having at least two different cross sections.

40. The optical waveguide according to claim 30, wherein the optical transmission, defined as the ratio I2/I1 of the intensity of the outcoupled light to the intensity of the incoupled light is greater than 50%, for incoupled light having wavelengths between 180 nm and 25 µm, said optical waveguide having a length smaller than 2 m.

41. The optical waveguide according to claim 40, wherein said optical transmission, is greater than 80% for incoupled light having wavelengths between 300 nm and 5 µm.

42. The optical waveguide according to claim 30 configured to be elastically stretchable up to at least 10% of the optical waveguide’s length and so that, after having been stretched, the optical transmission remains at least 90% of the transmission of the optical waveguide before being stretched.

43. The optical waveguide according to claim 30, comprising at least two core layers.

44. An optical waveguide bundle comprising at least three optical waveguides according to claim 30.

45. A medical device comprising at least one optical waveguide according to claim 30.

46. The medical device according to claim 44, wherein said device is a cochlear implant.

47. An optical sensor comprising at least one optical waveguide according to claim 30 and an optical cavity sensor head arranged to said optical waveguide, the sensor head comprising an optical cavity closed by flexible membrane.

48. A method of fabrication of an optical waveguide according to claim 30 comprising the steps (A-D) of:
A) realizing a hollow preform made of a thermoplastic elastomer
B) reducing the diameter of said preform and elongating said preform until a capillary is formed having a predetermined length and a predetermined cross section, said capillary having a central opening having a predetermined cross section
C) introducing liquid silicone into the central opening of said preform;
D) polymerising said liquid silicone so as to form an optical waveguide having a core being made of polymerised liquid silicone.

49. The method of fabrication according to claim 48, wherein step C and D are replaced by the steps E to G:
E) introducing liquid silicone during said step B of reducing the diameter of said preform;
F) while reducing the diameter of said preform keeping said liquid silicone in a liquid state until a predetermined length and a predetermined cross section of a precursor optical waveguide is obtained;
G) thermal polymerising said liquid silicone and said capillary so as to form an optical waveguide.

50. The method of fabrication according to claim 48, wherein step B, C and D are replaced by the steps H to J:
H) after said step A, introducing a liquid polymer into the central aperture of said hollow preform;
I) reducing the diameter of the preform filled with liquid polymer and elongating said filled preform until a capillary is formed filled with liquid polymer , said capillary having a predetermined length and a predetermined cross section;
J). polymerising said liquid polymer by applying UV light.

51. The method according to claim 50, wherein said liquid polymer is liquid silicone.

52. The method according to claim 50, wherein said liquid polymer is liquid siloxane.

53. The method of fabrication according to claim 48, wherein said obtained optical waveguide is a multimode or a monomode optical fiber.

54. The method of fabrication according to claim 48, wherein said obtained waveguide is a multicore optical fiber.

55. The method of fabrication according to claim 48, wherein said obtained waveguide is an optical waveguide having a non-circular cross section defined in any lateral plane.

56. The method of fabrication according to claim 48, wherein said thermoplastic is one of: a styrenic block copolymer, thermoplastic polyolefinelastomers, thermoplastic Vulcanizate, thermoplastic polyurethanes, thermoplastic copolyester, thermoplastic polyamides or not classified thermoplastic elastomers,, said thermoplastics being defined according to the ISO norm 18064.

57. A method of performing a surgical operation comprising: providing the optical waveguide according to claim 30, providing a chirurgical instrument, and using the optical waveguide in association with the chirurgical instrument during the surgical operation.

58. The method of claim 57, comprising tracking of the localisation of the position of said chirurgical instrument and/or the tracking of optical properties of tissues in the neighbourhood of the tip of said chirurgical instrument.
